(19) United States
(12) Patent Application Publication
(10) Pub. No.: US 2004/0208932 A1
(43) Pub. Date: Oct. 21, 2004

(54) Stabilized paroxetine hydrochloride formulation

(76) Inventors: Ramachandran Thembalath, Mumbai (IN); Yatish Kumar Bansal, Mumbai (IN); Veena Singh, Mumbai (IN)

Correspondence Address: PHARMACEUTICAL PATENT ATTORNEYS, LLC, 55 MADISON AVENUE, 4TH FLOOR, MORRISTOWN, NJ, 07960-7397, US

(21) Appl. No.: 10/768,348

(22) Filed: Jan. 30, 2004

(30) Foreign Application Priority Data
Apr. 17, 2003 (IN) 384/MUM/2003
Sep. 18, 2003 (IN) 977/MUM/2003
Oct. 31, 2003 (WO) PCT/IN03/00349

(51) Int. Cl. A61K 9/24

(52) U.S. Cl. 424/471

(57) ABSTRACT

A stabilized oral dosage form of an active pharmaceutical ingredient (API) such as paroxetine hydrochloride for improving the stability of the said API prior to incorporating into an oral delivery system, and a process for preparation of free flowing granules of paroxetine hydrochloride obtained by coating them with moisture barrier pharmaceutical excipients.

### GOVERNMENT INTEREST

None.

### RELATED APPLICATIONS

This application claims priority from India Provisional Application No. 348/MUM/2003, filed 16 Apr. 2003; India Utility Patent Application No. 976/MUM/2003, filed 18 Sep. 2003; India Utility Patent Application No. 977/MUM/2003, filed 18 Sep. 2003; and PCT Application No. PCT/IN03/______, filed 31 Oct. 2003.

### BACKGROUND

This invention relates to novel pharmaceutical preparations and a process of production thereof. More specifically, the invention relates to a novel process of preparing a stabilized oral dosage form of an active pharmaceutical ingredient (API) such as paroxetine hydrochloride and a novel process for improving the stability of the said active pharmaceutical ingredient (API) prior to incorporating into an oral delivery system. This invention further relates to a process for preparation of free flowing granules of paroxetine hydrochloride obtained by coating them with moisture barrier pharmaceutical excipients. More specifically, this invention relates to the process for the preparation of coated granules of paroxetine hydrochloride anhydrate and oral pharmaceutical compositions containing the same.

Paroxetine is chemically described as (-)-trans-4-((4′-flurophenyl)3-3(3′4′-methylenedioxy phenoxy methyl)-piperidine. Paroxetine has been approved for treating depression in humans.

Paroxetine (API) has first been claimed for its antidepressant properties in U.S. Pat. No. 3,912,743 and U.S. Pat. No. 4,007,196 (Ferrosan, Denmark). In 1980 paroxetine was licensed to Smithkline, where paroxetine was described as the maleate salt.

Crystalline paroxetine hydrochloride hemihydrate, process for its preparation, compositions containing the same and its preparation, and its herapeutic use as antidepressant has been claimed in U.S. Pat. No. 4,721,723 and EP 223403.

Thereafter, a large number of patent applications have been filed and patents granted for different forms of the API different pharmaceutical formulations using paroxetine and processes for formulating the same.

Patent WO9958113 describes paroxetine hydrochloride used in amorphous form or in the form of a crystalline anhydrate which is formulated into tablets under conditions such that there is no detectable conversion b hemihydrate during the tabletting process. Such conditions have been achieved by the use of essentially anhydrous or low moisture excipients such as dibasic calcium phosphate anhydrous (A_TAB*), anhydrous direct compression lactose, monosachharide sugars e.g. mannitol, disaccharide sugars e.g. lactitol (Finlac DC*), powdered cellulose, pregelatinised starch, microcrystalline cellulose (Avicel PH112*), sodium starch glycolate, croscarmellose sodium(Ac-Di-SolF*),colloidal silicon dioxide (Syloid 244*) (Explotab*), magnesium stearate and talc. Paroxetine hydrochloride anhydrate is mixed with the anhydrous or low moisture excipients and compressed using standard pharmaceutical procedures. As an additional aid to the protection of this product from the deleterious affects of moisture, the tablets are film-coated using hydrophobic coating materials such as glyceryl behenate (Compitrol 888*) using a hot melt coating technique.

Patent WO9958116 uses the same API and excipients for a capsule formulation i.e. paroxetine hydrochloride anhydrate is mixed with anhydrous or low moisture excipients and filled into cellulose capsule shell of intrinsically low moisture content (e.g. Shiono Qualicaps). The invention also finds that dibasic calcium phosphate anhydrous and polyglycolized glycerides can be used to form oral swallow capsules with paroxetine anhydrate without undesired conversion to hemihydrate during manufacturing process.

Patent WO02102382 describes a process for preparing paroxetine hydrochloride from paroxetine base which provides paroxetine hydrochloride substantially free of pink-colored compounds or an impurity identified by an HPLC RRT of about 1.5.

U.S. Pat. No. 5,955,475 describes an invention where paroxetine free base is formulated into pharmaceutical compositions when adsorbed on or absorbed by a solid carrier.

Patent WO 9831365 elaborates a process for preparing a free flowing form of paroxetine hydrochloride which comprises spray drying a solution of paroxetine hydrochloride. However no discussion appears in the patent regarding the problem of colour development.

U.S. Pat. No. 6,168,805 discloses an invention that relates to a process for preparing solid, amorphous paroxetine comprising a) mixing paroxetine free base or its salt with water and a pharmaceutically acceptable polymer and b) drying to form a composition comprising amorphous paroxetine and polymer, eliminating the need for organic solvents common for the solvent process. The resultant amorphous solid paroxetine composition is free from crystalline form and yet has good handling properties, making it suitable for pharmaceutical use in the traditional tablet dosage form.

Patent WO0102393 complexes of paroxetine, as free base or salt, with cyclodextrin or a cyclodextrin derivative show a high chemical stability, an improved solubility in water and are suitable for the preparation of liquid or solid pharmaceutical compositions.

Patent WO9948499 paroxetine free base is advantageously formulated into pharmaceutical compositions when adsorbed on or absorbed by a solid carrier. The composition of this invention is simply obtained by combining a solution of paroxetine with a suitable adsorbent or absorbent material and evaporating the solvent, for example by spray drying.

U.S. Pat. No. 6,503,927 describes a stable amorphous paroxetine hydrochloride composition employing an aqueous solvent medium containing an acidulant and polyvinylpyrrolidone and drying the resulting solid dispersion. The preferred compositions include amorphous paroxetine hydrochloride, polyvinylpyrrolidone and citric acid.

WO9926625 provides pharmaceutical formulations of paroxetine in which paroxetine is in solution in a solid, semi-solid or liquid carrier. The solutions are used to fill capsules, or self-supporting solid solutions are shaped into solid dosage forms such as tablets or pellets.

Patent WO 95/16448 reveals that earlier commercial paroxetine hydrochloride hemihydrate tablets were made using a wet granulation process. Further, the commercial tablets exhibited a colour change i.e. the tablets developed a pink hue that is undesirable.

Patent US2002065301 elaborates paroxetine salt compositions made with the aid of water by controlling the pH to 6.5 or less. These compositions have improved stability without significant coloration problems. The paroxetine salts include paroxetine hydrochloride salts but preferably use paroxetine sulfonate salts such as paroxetine methane sulfonate.

U.S. Pat. No. 6,113,944 relates paroxetine which is formulated into tablets using a formulation process in which water is absent. Direct Compression technique has been used where paroxetine hydrochloride hemihydrate is conventionally admixed with dry excipients and compressed into tablets or by dry granulation techniques as in U.S. Pat. No. 6,007,842 where paroxetine hydrochloride hemihydrate is conventionally admixed with dry excipients and compressed into large slugs or roller compacted into ribbon-like strands. The compacted material is then suitably milled to produce a free flowing powder which is then compressed into tablets. The excipients revealed in the patent include dicalcium phosphate dihydrate (Emcompress* or Ditab*), microcrystalline cellulose (Avicel PH 102*), sodium starch glycollate (Explotab*) & magnesium stearate.

### SUMMARY

We have provided a novel pharmaceutical preparation and a process for production thereof, the active pharmaceutical ingredient being formulated with a protective coating prior to incorporating into the dosage form. We have thereby substantially eliminated the possibility of degradation or color development by accelerated stability studies and have introduced characteristics of stability into the solid oral dosage form.

In accordance with the present invention, there is provided a substantially moisture stable pharmaceutical preparation in the form of a solid oral dose comprising;

(a) an active core comprising a granulated pharmaceutically active ingredient; and

(b) a moisture barrier coating enveloping individual granules of the active core.

Preferably, the moisture barrier coating permeates the active core, enveloping individual granules of the core. Even more preferably, granules in the region of the center of the active core are surrounded with and contacted by the moisture barrier coating. Accordingly, the invention provides a substantially moisture stable pharmaceutical preparation in the form of a solid oral dose comprising;

(a) an active core comprising a granulated pharmaceutically active ingredient; and

(b) a barrier coating surrounding the active core comprising a moisture barrier agent dispersed in an organic solvent.

“Substantially moisture stable” means that the preparation has the ability to retard degradation by means of water.

The usage of ethylcellulose provided a hydrophobic coating to the active and improved the stability of the product by inhibiting oxidation. Ethylcellulose additionally worked as a binder in the formulation. Granules coated with ethylcellulose demonstrated the added advantage of ability to absorb compression pressure and hence protect the coating from breaking during compression.

Coated granules of paroxetine hydrochloride anhydrate are disclosed which are prepared using a solution of moisture barrier excipient and a nonionic surfactant in an organic solvent. Such granules are manufactured by preparing a semisolid mass of the API and the solution of moisture barrier coating, preparing strands of suitable diameter of the wet mass, drying the strands and finally milling to get granules of desired size. The granules of the API are then incorporated into solid oral dose formulations of paroxetine. Alternately the coating of powder is obtained by coating fluidized API in a suitable equipment.

In accordance with a further aspect of the present invention, there is provided a process for producing a substantially moisture stable pharmaceutical preparation in the form of a solid oral dose as described hereinabove comprising the steps of:

(a) granulated a pharmaceutically active ingredient to form a granulated active core;

(b) coating the individual granules of the active core with a barrier coating comprising a moisture barrier agent; and

(c) forming the coated granules into a solid oral dose.

Thus, the invention provides a process for producing a substantially moisture stable pharmaceutical preparation in the form of a solid oral dose comprising the steps:

(a) granulated a pharmaceutically active ingredient to form an active core ; and

(b) coating the active core with a barrier coating comprising a moisture barrier agent dispersed in an organic solvent.

### DETAILED DESCRIPTION

In keeping with our objective of providing long term stability to the oral solid dosage form of paroxetine hydrochloride, we have selected excipients which would contribute to this characteristic objective. We have chosen not to use excipients such as disaccharides such as maltose, lactose, sucrose and glucose. Solvents like water or any other aqueous solvent or solvents that are freely miscible with water have also not been used.

We have also considered a coating agent which would provide excellent protection against moisture and at the same time immediately release the drug in the gastro-intestinal environment, as desired.

Paroxetine hydrochloride anhydrous has been chosen for experimental trials since it is considered more difficult to protect from moisture. It is also an aspect of the present invention to provide a pharmaceutical composition incorporating paroxetine hydrochloride hemihydrate by using the process herein above.

The process has also provided positive results with regard to other moisture barrier excipients such as polyethylene glycols, polyglycolised glycerides, fatty alcohols, stearic acid, opadry AMB OY-B-28920 white and Opadry 20A 58900 white, fatty materials of plant and animal origin. Additionally the tablets may also be film coated with hydrophobic coating materials to help retard against degradation.

The following examples illustrate the various aspects of the present invention.

### EXAMPLE 1

A coating solution of ethylcellulose was produced to dissolve in methylene chloride and iso-propyl alcohol. Polysorbate was added to this solution. The active was coated with this coating solution. The coated granules formed were dried at a suitable temperature and screened through a mesh of appropriate size. Dicalcium phosphate, microcrystalline cellulose and sodium starch glycollate were milled to which milled citric acid was geometrically mixed. Finally the dried mass of coated active granules were sized appropriately and blended with the above mixture and lubricated with the help of magnesium stearate. These resultant granules could be adequately compressed to tablets or could be suitably filled into hard gelatin capsule shells.

The pharmaceutical composition of the tablets containing paroxetine hydrochloride anhydrous has the following composition.

| | | |
|---|---|---|
| Paroxetine hydrochloride anhydrous | 33.32 | mg |
| Polysorbate 80 | 2.00 | mg |
| Ethylcellulose (10 cps) | 0.33 | mg |
| Acetone; Isopropyl alcohol | 1:3 | ratio |
| Dicalcium phosphate (dihydrate granular) | 320.35 | mg |
| Microcrystalline cellulose (Avicel PH 102) | 100.00 | mg |
| Sodium starch glycollate (Primogel) | 20.00 | mg |
| Citric acid | 4.00 | mg |
| Magnesium sterate | 5.00 | mg |

### EXAMPLE 2

The moisture retardant coated active pharmaceutical ingredient was prepared by Fluid Bed Processor (GLATT).

Ethyl cellulose was dissolved in the solvent mixture of methylene chloride and isopropyl alcohol. Complete dissolution was ensured and then polysorbate 80 was added to the solution and mixed avoiding foaming.

The bowl of the Fluid bed processor (FBP) was loaded with paroxetine hydrochloride anhydrate. The API was fluidized in the FBP and coating solution sprayed through the spray nozzle till granulation point was reached which was confirmed at the entrance port on the exterior of the expansion chamber.

Inlet temp. 60° C.-80° C.

Product temp. 30° c.-45° C.

Flap opening 25%-50%

Spray rate 10%-20%

Atomising air NLT 2.5 Kg/cm2

pressure

(iv) The granules were dried to a desired moisture content of NMT 1%

(v) Dicalcium phosphate (dihydrate granular) was added, microcrystalline cellulose (Avicel pH 102), sodium starch glycollate (Primogel), milled citric acid anhydrous and fluidised. Magnesium sterate was added and further fluidized.

(vi) The blend was compressed into tablets using suitable punches.

(vii) The tablets are aqueous film coated using HPMC

### EXAMPLE 3

Alternately, the active ingredient was coated by a moisture barrier solution and granulated by Rapid Mixer Granulator (RMG).

(i) Coating solution preparation

Ethyl cellulose was dissolved in the solvent mixture of methylene chloride and isopropyl alcohol. Complete dissolution was ensured and polysorbate 80 was added in the solution and mixed avoiding foaming.

(ii) The bowl of the Rapid Mixer Granulator (RMG) was loaded with paroxetine hydrochloride anhydrate. The mixer was started at low speed. The coating solution was poured on the bed of the paroxetine hydrochloride powder and mixed till a wet mass was obtained. The wet mass was sized using suitable screens.

(iii) The granules were dried in a fluid bed drier with the following parameters till the moisture content of NMT 1%

Inlet temp. 60° C.-70° C.

Product temp. 30° C.-45° C.

(iv) Dicalcium phosphate (dihydrate gamular), microcrystalline cellulose (Avicel pH 102), sodium starch glycollate (Primogel) and citric acid anhydrous were added and mixed in a double cone blender. Magnesium sterate was added and mixed thereafter.

(v) The resultant blend was compressed into tablets using suitable punches.

(vi) The tablets were aqueous film coated using HPMC

Consulsion

Although this invention has been described with reference to specific embodiments thereof, it is to be understood that other embodiments and variations of the inventions as described and exemplified may be made by those skilled in the art without departing from the true spirit of invention. We therefore intend the coverage of our patent be defined not by the specific examples we discuss here, but by the following claims.

We claim:

1. A substantially moisture stable pharmaceutical preparation in the form of a solid oral dose comprising;
(c) an active core comprising a granulated pharmaceutically active ingredient; and
(d) a moisture barrier coating enveloping individual granules of the active core.

2. A pharmaceutical preparation as claimed in claim 1, wherein the ndisture barrier coating permeates the active core, enveloping individual granules of the core.

3. A pharmaceutical composition according to claim 2, wherein granules in the region of the center of the active core are surrounded with and contacted by the moisture barrier coating.

4. A pharmaceutical preparation as claimed in claim any preceding claim, wherein the active pharmaceutical ingredient is paroxetine hydrochloride anhydrate or paroxetine hydrochloride hemihydrate.

5. A pharmaceutical preparation as claimed in any preceding claim, wherein the barrier coating is hydrophobic.

6. A pharmaceutical preparation as claimed in any preceding claim, wherein the barrier coating further comprises a nonionic surfactant.

7. A pharmaceutical preparation as claimed in any preceding claim, wherein the barrier coating comprises a moisture barrier agent selected from one or more of the following agents: ethyl cellulose, polyethylene glycols, polyglycolised glycerides, fatty alcohols, stearic acid, opadry AMB OY-B-28920 white and Opadry 20A 58900 white and fatty materials of plant and animal origin.

8. A pharmaceutical preparation as claimed in any preceding claim, incorporating anhydrous citric acid for pH related stability adjustment.

9. A pharmaceutical preparation as claimed in any preceding claim, further comprising one or more of the following ingredients: a diluent, a disintegrant and a lubricant.

10. A pharmaceutical preparation as claimed in claim 9, wherein dibasic calcium phosphate or microcrystalline cellulose is used as a diluent.

11. A pharmaceutical preparation as claimed in any one of claims 8 to 10, wherein sodium starch glycollate is used as a disintegrant.

12. A pharmaceutical preparation as claimed in any of claims 8 to 11, wherein magnesium stearate is used as a lubricant.

13. A pharmaceutical preparation as claimed in preceding claim, wherein the preparation is in the form of a tablet or the preparation is placed within a capsule.

14. A pharmaceutical preparation as claimed in claim 13, wherein the tablet is caplet shaped.

15. A pharmaceutical preparation as claimed claim 13 or claim 14, wherein the granules are compressed into tablets with hardness ranging from 150-200 Norton

16. A pharmaceutical preparation as claimed in any of claims 13 to 15, wherein the tablets are optionally further coated with conventional film coating materials.

17. A pharmaceutical preparation as claimed claim 16, wherein the film coating is a hydrophobic material.

18. A pharmaceutical preparation as claimed in any preceding claim, wherein the pharmaceutical preparation is substantially resistant to moisture-degradation of the active ingredient and/or the development of pink hue.

19. A pharmaceutical preparation as claimed in any preceding claim, wherein the pharmaceutical preparation further comprises pharmaceutically acceptable excipients in order to mask the taste of the preparation.

20. A pharmaceutical preparation as claimed in any of claims 11 to 12 and 17 to 18, wherein the preparation is placed into hard gelatin capsules

21. A process for producing a substantially moisture stable pharmaceutical preparation in the form of a solid oral dose as described in any one of claims 1 to 19 comprising the steps of:
(d) granulated a pharmaceutically active ingredient to form a granulated active core;
(e) coating the individual granules of the active core with a barrier coating comprising a moisture barrier agent; and
(f) forming the coated granules into a solid oral dose.

22. A process according to claim 21, wherein the coating is achieved by contacting individual granules of the active core with a solution of the moisture barrier agent in an organic solvent.

23. A procees according to claim 22, wherein the contacted granules are dried to remove the organic solvent and provide individual coated granules.

24. A process according to claim 22 or claim 23 wherein the organic solvent is selected from methylene chloride, isopropyl alcohol, acetone and mixtures of one or more thereof.

25. A process according to claim 24, wherein Polysorbate 80 is added to the organic solvent.
